(19) United States
(12) Patent Application Publication
(10) Pub. No.: US 2015/0119671 A1
(43) Pub. Date: Apr. 30, 2015

(54) SYSTEM AND METHOD FOR IDENTIFYING A MECHANISM OF ACTION OF AN ARRHYTHMIA

(71) Applicant: THE CLEVELAND CLINIC FOUNDATION, Cleveland, OH (US)

(72) Inventor: Niraj Varma, Cleveland Hts., OH (US)

(21) Appl. No.: 14/526,949

(22) Filed: Oct. 29, 2014

Related U.S. Application Data
(60) Provisional application No. 61/896,736, filed on Oct. 29, 2013.
(60) Provisional application No. 61/990,787, filed on May 9, 2014.

(51) Int. Cl. A61B 5/04; A61B 18/02; A61B 5/0452; A61B 18/20; A61B 18/18; A61B 5/042; A61B 18/12; A61N 7/00

(52) U.S. Cl. CPC A61B 5/04011; A61B 18/12; A61B 18/02; A61N 7/00; A61B 18/20; A61B 18/1815; A61B 5/0422; A61B 5/0452; A61N 2007/0004; A61B 2018/00577
USPC 600/374; 606/32; 606/21; 606/169; 606/2; 606/33

(57) ABSTRACT

Systems and methods are provided that can identify a mechanism of action of an arrhythmia in a chamber of the heart. A plurality of electrograms recorded by a plurality of electrodes contacting a wall of a chamber of the heart at a corresponding plurality of different locations within the chamber in response to an electrical perturbation can be received. An activation map of the chamber can be determined based on the plurality of electrograms. Based on the activation map, a location and a shape of a mechanism of action of the arrhythmia can be determined. A treatment plan can be developed based on a location and a size of the mechanism of action within the chamber. For example, the treatment plan can include guiding an ablation of the mechanism of action.

### CROSS-REFERENCE TO RELATED APPLICATIONS

This application claims the benefit of U.S. Provisional Patent Application No. 61/896,736, filed Oct. 29, 2013, entitled SYSTEM AND METHOD OF IDENTIFYING A DRIVING CIRCUIT OF AN ARRHYTHMIA, and U.S. Provisional Patent Application No. 61/990,787, filed May 9, 2014, entitled SYSTEM AND METHOD FOR TREATING UNSTABLE ARRHYTHMIAS. The subject matter of these applications is incorporated herein by reference in their entirety.

### FIELD OF THE INVENTION

The present disclosure relates generally to the identification of a mechanism of action of an arrhythmia and, more specifically, to systems and methods that can identify the mechanism of action of the arrhythmia based on electrical activity of a chamber of the heart.

### BACKGROUND

Generally, an arrhythmia can refer to an abnormality in the timing or pattern of electrical conduction in the heart. For example, an arrhythmia can cause the heart to beat too rapidly, too slowly, or irregularly. One method that can be used to treat arrhythmias is catheter ablation, in which energy can be delivered through a catheter to tiny areas of the heart muscle. This energy can promote normal conduction by disconnecting the pathway of the abnormal rhythm, blocking the abnormal pulses, and/or disconnecting the electrical pathway between the atria and ventricles. However, some types arrhythmias are difficult to ablate, resulting in patient morbidity and physician frustration.

One type of arrhythmia that is difficult to ablate a stable arrhythmia with a complex mechanism of action (e.g., due to the electrophysiological phenomenon of reentry, which can occur when an electrical impulse fails to die out after normal activation of the heart due to abnormal conduction within the heart). Examples of arrhythmias with such complex mechanisms of action can include: atrial flutter, atrioventricular (AV) nodal reentry, ventricular tachycardia and ventricular fibrillation. Another type of arrhythmia that is difficult to ablate is an unstable arrhythmia that depends on the dynamic interplay between multiple driving circuits. Examples of such unstable arrhythmias can include: persistent atrial fibrillation (AF) and chronic AF.

### SUMMARY

The present disclosure relates generally to the identification of a mechanism of action of an arrhythmia. More specifically, the present disclosure relates to systems and methods that can identify the mechanism of action of the arrhythmia based on electrical activity of a chamber of the heart and create a treatment plan for the arrhythmia based on the location of the mechanism of action.

In one aspect, the present disclosure can include a catheter for insertion into a patient's heart (e.g., into a chamber of the patient's heart). The catheter can include an elongated body with a distal end that includes an expansion element that has a plurality of electrodes. The expansion element can be configured to expand within a chamber of the heart in a shape corresponding to the shape of the chamber. This allows the plurality of electrodes to contact a wall of the chamber to enable a characterization of a mechanism of action underlying an arrhythmia from an activation map created based on a plurality of electrograms recorded by the plurality of electrodes in response to an electrical perturbation to the chamber.

In another aspect, the present disclosure can include a method. One or more acts of the method can be represented by computer-executable instructions that can be stored in a non-transitory memory and executed by a system comprising a processor. The acts can include: receiving a plurality of electrograms recorded by a plurality of electrodes contacting a wall of a chamber of the heart at a corresponding plurality of different locations within the chamber in response to an electrical perturbation to the chamber; determining an activation map of the chamber based on the plurality of electrograms; determining a location and a shape of a mechanism of action of the arrhythmia based on the activation map; and guiding an ablation of the mechanism of action based on the location and the shape of the mechanism of action.

In a further aspect, the present disclosure can include a system that can include a non-transitory memory to store machine-executable instructions and a processor to access the non-transitory memory and execute the machine-executable instructions to cause a computing device to perform operations. A plurality of electrograms recorded by a plurality of electrodes contacting a wall of a chamber of the heart at a corresponding plurality of different locations within the chamber in response to an electrical perturbation to the chamber can be received. An activation map of the chamber can be determined based on the plurality of electrograms. Based on the activation map, a shape and a location of a mechanism of action of the arrhythmia can be determined. An ablation of the mechanism of action can be guided based on the location and the shape of the mechanism of action.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a system block diagram depicting a system that identifies a mechanism of action of an unstable arrhythmia and develops a treatment plan to abolish the mechanism of action in accordance with an aspect of the present disclosure;

FIG. 2 is a schematic diagram depicting a cross section of a catheter with an expandable element in an unexpanded state in accordance with an aspect of the present disclosure;

FIG. 3 is a schematic diagram depicting a cross section of the catheter with the expandable element in an expanded state in accordance with an aspect of the present disclosure;

FIG. 4 is a schematic diagram depicting a cross section of the catheter with the expandable element in an unexpanded state before activation of an actuating wire by a control mechanism in accordance with an aspect of the present disclosure;

FIG. 5 is a schematic diagram depicting a cross section of the catheter with the expandable element in an expanded state after activation of the actuating wire by the control mechanism in accordance with an aspect of the present disclosure;

FIG. 6 is a schematic diagram depicting a cross section of the expandable element in the expanded state in a chamber of the heart with a plurality of pacing electrodes located within the expandable element in accordance with an aspect of the present disclosure;

FIG. 7 is a schematic diagram depicting a cross section of the expandable element in the expanded state in the chamber of the heart with the plurality of pacing electrodes located in a contralateral chamber in accordance with an aspect of the present disclosure;

FIG. 8 is a schematic diagram depicting a cross section of the catheter with the expandable element in the expanded state and an ablation mechanism to deliver an ablation sequence determined according to the treatment plan in accordance with an aspect of the present disclosure;

FIG. 9 is a process flow diagram depicting a method for developing a treatment plan to abolish the mechanism of action of an arrhythmia in accordance with an aspect of the present disclosure;

FIG. 10 is a process flow diagram depicting a method for identifying and treating a mechanism of action of an arrhythmia in accordance with another aspect of the present disclosure; and

FIG. 11 is a system block diagram depicting an exemplary system of hardware components capable of implementing examples of the system of FIG. 1 and/or the methods of FIGS. 9 and 10.

### DETAILED DESCRIPTION

### I. Definitions

In the context of the present disclosure, the singular forms “a,” “an” and “the” can also include the plural forms, unless the context clearly indicates otherwise. The terms “comprises” and/or “comprising,” as used herein, can specify the presence of stated features, steps, operations, elements, and/or components, but do not preclude the presence or addition of one or more other features, steps, operations, elements, components, and/or groups. As used herein, the term “and/or” can include any and all combinations of one or more of the associated listed items. Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “arrhythmia” can refer to a condition in which the electrical conduction in the heart is abnormal. For example, an arrhythmia can cause the heart to beat too rapidly, too slowly, or irregularly. Arrhythmias can be stable and/or unstable. Examples of stable arrhythmias can include: atrial flutter, atrioventricular (AV) nodal reentry, ventricular tachycardia and ventricular fibrillation. Examples of stable arrhythmias can include: persistent atrial fibrillation (AF) and chronic AF.

As used herein, the terms “mechanism of action” can refer to areas of abnormal conduction in chamber of a patient's heart that contribute to the genesis of an arrhythmia. The mechanism of action can have one or more driving circuits. For example, a stable arrhythmia can have a single driving circuit, which can be complex (e.g., reentry) as its mechanism of action, while an unstable arrhythmia can have a mechanism of action that relies on the dynamic interplay of two or more driving circuits.

As used herein, the term “dynamic” can refer to a property that is characterized by continuous change and/or evolution. For example, a dynamic property can vary with time.

As used herein, the term “electrical perturbation” can refer to a short electrical pulse that pings the arrhythmia. In some examples, an electrical perturbation can cause the arrhythmia to display its mechanism of action.

As used herein, the term “ablation” can refer to a procedure that uses a catheter to deliver energy to tiny areas of the heart muscle, scarring or destroying tissue in the heart contributes to the driving circuit, thereby promoting normal conduction.

As used herein, the term “electrogram” can refer to a record of a change in electrical potential in the heart recorded by one or more electrodes placed directly in or on the cardiac tissue. In some instances, the electrogram can be a graphical record that is made from the measurement of electrical events in the cardiac tissue. A whole chamber activation map can be created based on the plurality of electrograms.

As used herein, the term “patient” can refer to any warm-blooded organism, including, but not limited to, human beings, pigs, rats, mice, dogs, goats, sheep, horses, monkeys, apes, rabbits, cattle, etc.

### II. Overview

The present disclosure relates generally to the identification of a mechanism of action of an arrhythmia. For example, the mechanism of action can include one or more driving circuits of the arrhythmia. More specifically, the present disclosure relates to systems and methods that can identify the mechanism of action of the arrhythmia based on electrical activity of a chamber of the heart and create a treatment plan for the arrhythmia based on the location of the mechanism of action. For example, the treatment plan can include guiding an ablation of the mechanism of action. As an example, the treatment plan can be developed based on a comparison of the activation map to a plurality of stored historical activation maps. A closest proxy can be determined from the plurality of stored historical activation maps based on a pattern recognition technique and a proposed treatment plan can be developed based on a treatment plan of the closest proxy. The proposed treatment plan can be offered for approval, and, upon approval, can be selected as the treatment plan. The treatment plan and the activation map can be stored as one of the plurality of stored historical activation maps. The stored historical activation maps can also include data collected related to different mechanisms of action and corresponding ablative strategies.

### III. Systems

One aspect of the present disclosure can include a system that can identify a mechanism of action of an arrhythmia. Upon identification of the mechanism of action, the system can create a treatment plan to abolish the mechanism of action. For example, the system can guide an ablation of the arrhythmia based on the treatment plan. The treatment plan can be based on the location and the shape of the mechanism of action.

In some instances, the system can employ a catheter with an expandable element adapted to contact the chamber wall (e.g., made of a flexible material). The expandable element can include a plurality of electrodes that can contact the chamber wall upon expansion of the expandable element. The electrodes can record electrograms based on one or more electrical perturbations. The system can generate an activation map of the whole chamber of the heart based on the plurality of electrograms. In some instances, the activation map can be updated dynamically. The system can provide the ability of introducing an electrical perturbation and collecting responses to the perturbation from points across the chamber simultaneously.

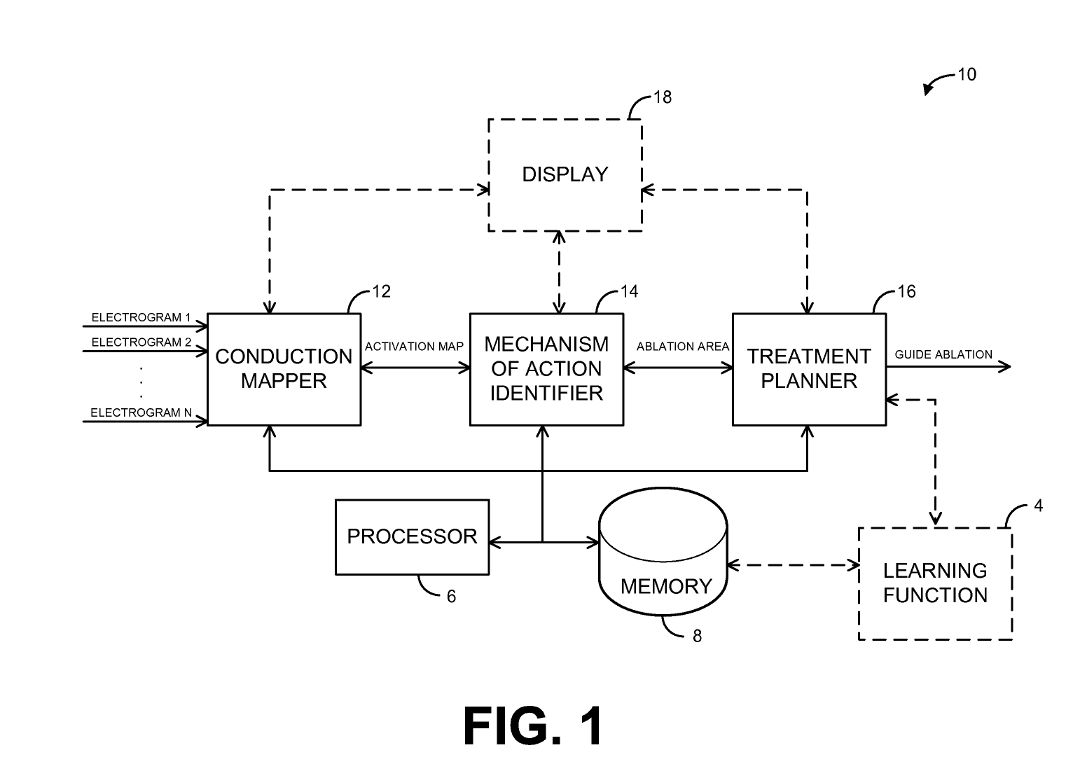

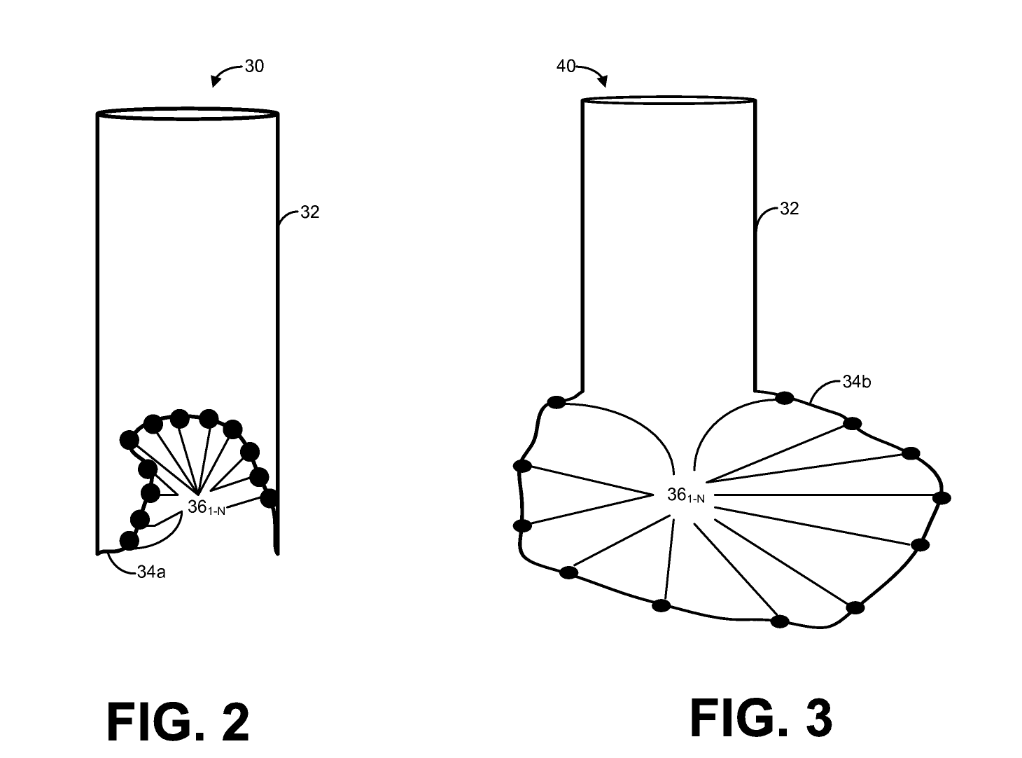

FIG. 1, as well as portions of the associated FIGS. 2-8, are schematically illustrated as block diagrams with the different blocks representing different components. The functions of one or more of the components can be implemented by computer program instructions. These computer program instructions can be provided to a processor of a general purpose computer, special purpose computer, and/or other programmable data processing apparatus to produce a machine, such that the instructions, which execute via the processor of the computer and/or other programmable data processing apparatus, create a mechanism for implementing the functions of the components specified in the block diagrams.

These computer program instructions can also be stored in a non-transitory computer-readable memory that can direct a computer or other programmable data processing apparatus to function in a particular manner, such that the instructions stored in the non-transitory computer-readable memory produce an article of manufacture including instructions, which implement the function specified in the block diagrams and associated description.

The computer program instructions can also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions of the components specified in the block diagrams and the associated description.

Accordingly, one or more components described herein can be embodied at least in part in hardware and/or in software (including firmware, resident software, micro-code, etc.). Furthermore, aspects of the system 10 can take the form of a computer program product on a computer-usable or computer-readable storage medium having computer-usable or computer-readable program code embodied in the medium for use by or in connection with an instruction execution system. A computer-usable or computer-readable medium can be any non-transitory medium that is not a transitory signal and can contain or store the program for use by or in connection with the instruction or execution of a system, apparatus, or device. The computer-usable or computer-readable medium can be, for example but not limited to, an electronic, magnetic, optical, electromagnetic, infrared, or semiconductor system, apparatus or device. More specific examples (a non-exhaustive list) of the computer-readable medium can include the following: a portable computer diskette; a random access memory; a read-only memory; an erasable programmable read-only memory (or Flash memory); and a portable compact disc read-only memory.

Referring now to FIG. 1, depicted is a system 10 that can that identifies a mechanism of action of an unstable arrhythmia and develops a treatment plan to abolish the mechanism of action in accordance with an aspect of the present disclosure. The system can include components, including a conduction mapper 12, a mechanism of action identifier 14, and a treatment planner 16. The components can be stored in a non-transitory memory 8 and executed by processor 6 to facilitate the performance of operations associated with the components. Optionally, the system 10 can include a display 18 and/or a learning function 4 that can access historical data. One or more of the components can be implemented by computer program instructions that can be stored in a non-transitory memory 8 (e.g., an electronic, magnetic, optical, electromagnetic, infrared, or semiconductor system, apparatus or device) and provided to a processor 6 (e.g., a processor of a general purpose computer, special purpose computer, and/or other programmable data processing apparatus). The processor 6 can execute the instructions such that a computing device can implement the functions of one or more of the components.

For example, the conduction mapper 12 receive a plurality of electrograms (e.g., electrogram 1-electrogram N) recorded by a plurality of electrodes (e.g., corresponding to the number of electrograms) contacting a wall of a chamber of the heart at a corresponding plurality of different locations within the chamber in response to an electrical perturbation to the chamber. For example, the plurality of electrograms can correspond to different portions of the chamber of the heart. In some examples, the plurality of electrograms can overlap between different portions of the chamber of the heart.

Based on the plurality of electrograms, the conduction mapper 12 can determine an activation map of the chamber. The number of electrodes and/or electrograms (e.g., N) can be chosen so that the conduction mapper 12 can generate an activation map of the entire chamber of the heart. In some examples, N can be at least 8. In other examples, N can be at least 16. In still other examples, N can be at least 128. Each of the electrograms can trace the electrical potential within the chamber from the corresponding electrode position. In some instances, the conduction mapper 12 can determine a proper subset of the plurality of electrograms that can be used to determine the activation map (e.g., eliminating electrodes without good contact with the chamber wall). The different electrode positions (or “points”) for the plurality of electrodes can be chosen so the electrodes do not need to be moved and/or repositioned to ensure that conduction mapper 12 can determine the activation map in a clear and detailed manner (e.g., the electrode at each point can trace the path of conduction from a large portion of the heart without overlapping to a point that drains computational resources). The conduction mapper 12 can map the “whole” chamber of the heart without using a sequential contact mapping technique that can require the electrodes to be in contact with the wall of the chamber or to move around the chamber to capture electrograms with signals having an adequate fidelity to determine the mechanism of action from the activation map.

For example, the activation map can provides information related to electrical propagation velocity, information related to attenuation, information defining a zone of dead tissue, and/or information related to relative activation. Based on at least these properties, the activation map can reveal the mechanism of action of the arrhythmia. In some instances, the activation map can be a dynamic activation map that can change over time (e.g., based on two or more perturbations). For example, the dynamic activation map can illustrate the dynamic interplay of multiple driving circuits that can work in combination to create the mechanism of action.

In contrast to stable arrhythmias, which often depend on a single driving circuit as their mechanism of action, unstable arrhythmias depend on the dynamic interplay among multiple drivers as their mechanism of action. These intricate and changing interrelationships may change in a matter of seconds. Tracking these leads to identification of critical components, and hence directed (as opposed to empiric) ablation. The plurality of electrodes can provide the whole chamber electrograms with sufficient rapidity to track the dynamic interplay between the multiple drivers and permits application of algorithmic analysis to resolve passive from active circuit components, and, thus, target critical drivers. The whole chamber electrograms 22 can map complex electrical activity across the entire chamber of the heart rapidly with one shot (e.g., with single cycle temporal resolution). Mapping the complex electrical activity across the entire heart chamber in this detail can offer insight into depolarization patterns and identify responsible regions that initiate or sustain arrhythmias.

Traditional methods for mapping conduction in the entire chamber of the heart, like sequential contact mapping, can be time consuming and is prone to error at least due to moving references, patient movement, changes to the patient hydration state, etc., and are likely to increase the longer it takes to map the heart chamber and are unavoidable using a serial contact mapping strategy. The intrinsic limitations of the sequential contact technique makes it suitable for constructing electrogram maps, which are an important, but only a first order characterization of the heart chamber's depolarization patterns that cannot provide further insight into the driving circuits of arrhythmias. In contrast, the conduction mapper 12 can determine the plurality of electrogams corresponding to the whole chamber of the heart at substantially the same time. The electrograms can elucidate useful second order conduction parameters of the heart chamber to identify aberrant regions of tissue that are often the source of arrhythmias and other dead regions of the tissue. The mechanism of action identifier 14 can employ one or more algorithms to determine the sources of the arrhythmia, as well as the dead regions, to guide ablation more efficiently and effectively. Real time changes to the circuits and, consequently to continually guide ablation targets.

In one example, the electrodes can be located at different points in contact with the wall of the chamber at a known spacing apart from one another, about equidistant from one another in a configuration that closely matches the volume and shape of the chamber. By utilizing a large number of electrodes with known spacing, algorithms are employed to assess electrical activity including voltage attenuation, conduction velocity and other measures of tissue conduction correlated to specific regions of the chamber. The aggregate evaluation of parameters enables a whole chamber map revealing areas of inhomogeneity. Arrhythmias are often a consequence of abnormal or diseased/damaged/dead tissue. The abnormal tissue that corresponds to arrhythmiagenesis can be isolated from damaged/diseased/dead tissue. Their origins and driving circuits (with localization of their critical components) may be revealed by using a holistic, multi-parameter characterization scheme.

In some instances, the electrograms can be recorded by a plurality of electrodes located on or within an expandable element of a catheter. The expandable element can be adapted to contact the chamber wall (e.g., made of a flexible material). A catheter used for mapping can include an elongated body and a distal end of the body. The distal end of the body can have the expansion element that includes a plurality of electrodes. For example, the expansion element can be configured to expand within a chamber of the heart in a shape corresponding to the shape of the chamber so that the plurality of electrodes contact the wall of the chamber. For example, the expansion element can be larger than the shape of the chamber to ensure contact between the plurality of electrodes and the wall of the chamber. As another example, the expansion element can have a number of different intermediate expansion capabilities to account for variation between patients and/or between chambers. In some instances, the expansion element can be constructed from one or more flexible and/or malleable materials. As an example, the expansion element can be constructed from nitinol.

A non-limiting example of a device that can be used to provide the electrograms is a modified catheter device 32, as depicted in FIGS. 2-8. The modified catheter device 32 can include an elongated body with a distal end that includes a flexible or malleable expansion element 34a, 34b. The expansion element 34a, 34b can include the plurality of electrodes 36_{1-N} (where the number of electrodes corresponds to the number of electrograms). The expansion element can be adapted to expand within a chamber of the heart in a shape corresponding to the shape of the chamber so that at least a portion of the plurality of electrodes 36_{1-N} contact a wall of the chamber. In some instances, each of the electrodes 36_{1-N} can contact the wall of the chamber. For example, the expansion element can to be elastic and conformable to the chamber of the heart to minimize heart wall irritation.

The plurality of electrograms, recorded at various points against the wall of the chamber, can enable a characterization of a mechanism of action underlying an arrhythmia (e.g., characterized by parameters, such as size, location, and the like) from an activation map created based on the plurality of electrograms in response to an electrical perturbation to the chamber. For example, the plurality of electrograms can be recorded simultaneously during a small number of beats of the heart during the pacing sequence without requiring rotation of the expansion element. In some instances, the number of beats can be from 1 beat-50 beats. In other instances, the number of beats can be from 5 beats-25 beats. In still other instances, the number of beats can be between 10 beats-15 beats.

The expansion element 34a, 34b can expand within the chamber (e.g., as depicted in FIG. 3) to contact the wall of the chamber or contract when in transport to or from the chamber (e.g., as depicted in FIG. 2). According to an aspect, the expansion element 34a, 34b can include a frame (e.g., made of a flexible and malleable material, such as a polymer or a combination of a metal and the polymer; one example material is national). In some instances, the expansion element can include an inflatable element. For example, the expandable element can be expanded by inflation of the inflatable element with a fluid medium. In another example, the expandable element can be expanded upon a trigger by an actuator mechanism. In some instances, the expansion element is configured to expand to a plurality of intermediate sizes to provide flexible sizing to match a plurality of sizes of a plurality of chambers of the heart in a plurality of patients.

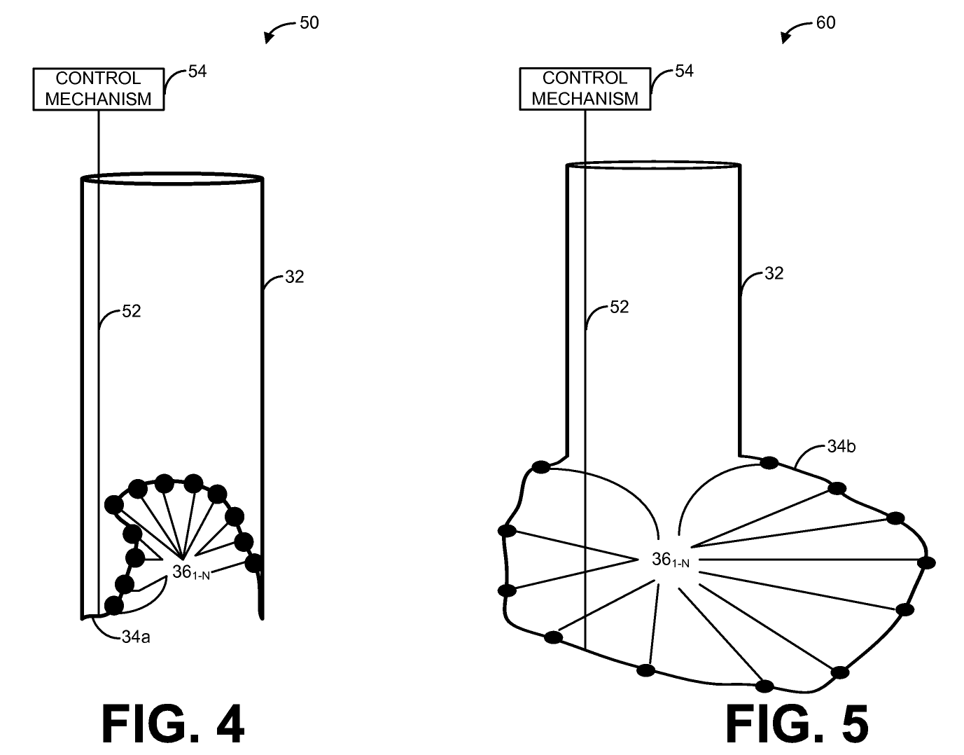

As depicted in FIGS. 4 and 5, the frame can be connected by an actuating wire 52 through the body of the modified catheter device 32 to a control mechanism 54 that can activate an expansion of the expansion element 34b (as shown in FIG. 4) or a contraction of the expansion element 34a (as shown in FIG. 5). In another aspect, the expansion element 34a, 34b can include an inflatable element that is expandable by inflation of the inflatable element with a fluid medium and contractible by deflation of the inflatable element (e.g., by removing the fluid medium).

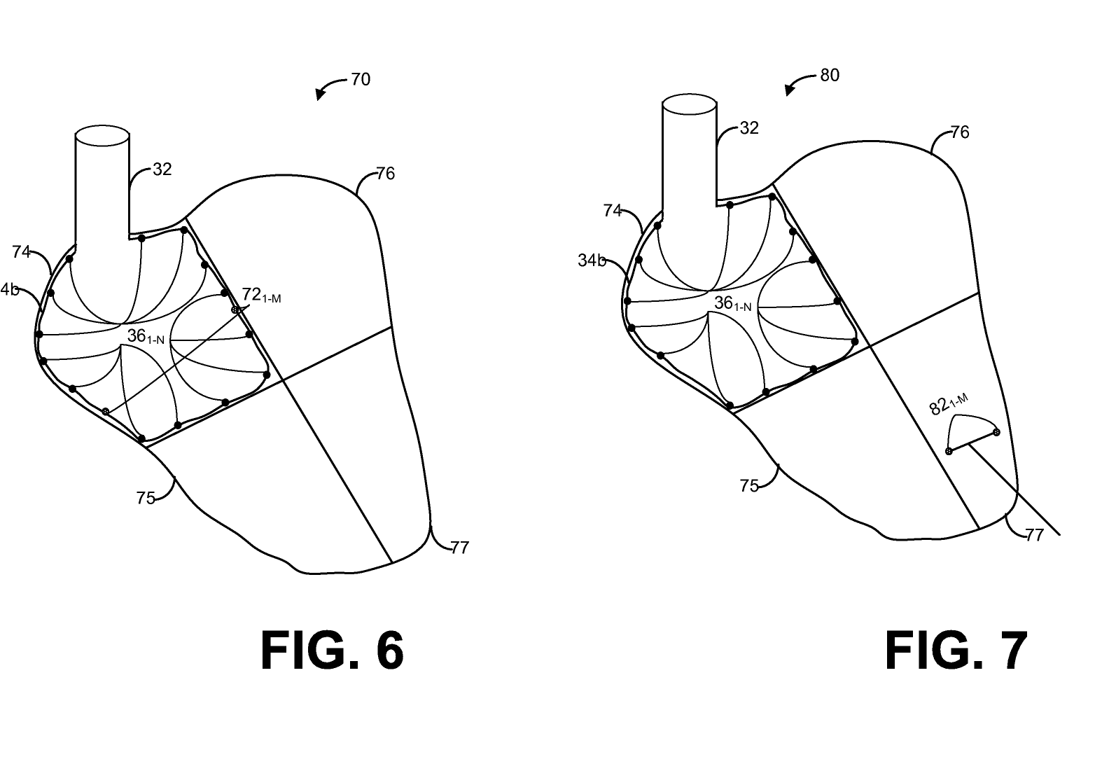

The shapes and configurations of the expansion element 34a, 34b as illustrated in FIGS. 2-8 are non-limiting examples shown for ease of illustration and explanation. It will be understood that the expansion element 34a, 34b can be of different shapes and configurations. As a non-limiting example, as depicted in FIGS. 6 and 7, the shape of the expanded expansion element 34b can be configured according to the shape of the chamber so that the plurality of electrodes 36_{1-N} do not need to be moved and/or repositioned during the recording of the electrograms.

FIGS. 6 and 7 illustrate examples of the expansion element 34b of the modified catheter device 32 expanded within in the heart 70, 80. The schematic diagrams of the heart 70, 80 each include four chambers: the right atrium 74, the right ventricle 75, the left atrium 76, and the left ventricle 77. It will be understood that the schematic diagrams of the heart 70, 80 are not necessarily drawn to scale and do not necessarily include all of the different anatomical features of the various chambers 74-77. Additionally, the electrodes of the expansion element 34b are meant to contact the wall of the chamber of the heart.

In FIGS. 6 and 7, the modified catheter device 32 is inserted into the right atrium 74 (e.g., through the vena cava) and the expansion element 34b is configured in a shape corresponding to the anatomical shape of the right atrium 74. It will be understood that the modified catheter device 32 can be inserted into any of the four chambers of the heart, and the expandable element 34b can be configured according to the shape of the corresponding chamber 74-77. In some instances, the expandable element 34b can be larger than the shape of the right atrium to ensure contact with the heart wall surface. In some instances, the expandable element 34b can be configured to be expanded to a plurality of different sizes corresponding to different chambers of different sizes. The modified catheter device 32 is not limited to insertion into the right atrium 74.

In the context of FIG. 1, the electrograms recorded by the electrodes (e.g., 36_{1-N} in FIGS. 2, 4, 6 and 3, 5, 7) in response to one or more electrical perturbations (e.g., a short pacing sequence). The modified catheter device 32 combined with a spatially pacing source (e.g., a regular pacing sequence or a dynamic pacing sequence) can provide conduction properties (size and location of the arrhythmia), conduction, velocity and voltage attenuation. These parameters can provide second order information helping to expose the mechanism of action of the arrhythmia. The one or more perturbations can be delivered to the chamber of interest (the right atrium 74 in FIGS. 6 and 7) according to a predefined pacing algorithm. The pacing algorithm can include a regular pacing sequence or a dynamic pacing sequence. According to a non-limiting example, as shown in FIG. 6, the pacing electrodes 72_{1-M} (where M is an even integer greater than or equal to two) can be positioned bilaterally within the chamber 74. Although FIG. 6 shows the pacing electrodes 72_{1-M} within the expandable element 34b, it will be understood that the pacing electrodes 72_{1-M} can be separate from the expandable element 34b. Alternatively, according to another non-limiting example, the pacing electrodes 82_{1-M} can deliver the pacing sequence from another chamber 77 of the heart located contralateral to the chamber 74 (e.g. the left ventricle 77 is located contralateral to the right atrium 74). Pacing electrodes 82_{1-M} can be located within the contralateral chamber (e.g., left ventricle 77) to provide the pacing or can be located external to the contralateral chamber 77 to provide the pacing.

Referring again to FIG. 1, conduction mapper 12 can determine the activation map based on the electrograms recorded from different areas within the chamber. For example, the conduction mapper 12 can employ one or more computational algorithms to convert the two dimensional data related to electrical conduction recorded by the electrodes to a three dimensional data that can be used to create the plurality of electrograms that is an electroanatomical reconstruction of the chamber. The conduction mapper 12 can send the activation map to the mechanism of action identifier 14.

The mechanism of action identifier 14 can determine a location and a shape of a mechanism of action of the arrhythmia based on the activation map. For example, the mechanism of action identifier 14 can utilize the information provided by the activation map as evidence of the size and the location of the mechanism of action. The mechanism of action identifier 14 can send a proposed ablation area including at least a portion of the mechanism of action to a treatment planner 16.

According to a non-limiting example, the mechanism of action identifier 14 that can apply one or more computational algorithms to the data related to the plurality of electrograms and/or the activation map to determine the location and configuration/shape of the mechanism of action within the chamber. The analysis can also determine one or more characteristics of the dynamically changing mechanism of action. The analysis of the data related to the plurality of electrograms and/or the activation map by the mechanism of action identifier 14 can illustrate dimensions and shape of the portions of the chamber that are involved in the arrhythmiagenesis of the driving circuit, while eliminating areas passively activated and those with scar/slow conduction that do not contribute to the arrhythmiagenesis. Based in the identification of the mechanism of action, the mechanism of action identifier 14 can provide a preliminary ablation area to the treatment planner 16. The preliminary ablation area can include at least a portion of the mechanism of action and/or be related to the mechanism of action.

In another non-limiting example, the plurality of electrograms and/or the activation map can be provided to a display 18 that can render an image of the conduction within the chamber that can allow the mechanism of action be visually displayed (e.g., in color). The mechanism of action identifier 14 can receive an input based on the rendered image that proposes the configuration of the mechanism of action. In this case, the mechanism of action identifier 14 can verify the proposed one or more locations of the mechanism of action before proposing the preliminary ablation area to the treatment planner 16.

The treatment planner 16 can determine a treatment plan to eliminate the mechanism of action of the arrhythmia. For example, the treatment can include catheter ablation. Accordingly, the treatment planner 16 can guide the ablation of the mechanism of action based on the treatment plan. For example, the treatment plan can take into account the location and the shape of the mechanism of action. In some examples, the ablation can be guided based on real time imaging and/or real time electrograms and/or the dynamically updated activation map.

The treatment planner 16 can develop a treatment plan to abolish the arrhythmia based on the mechanism of action. The treatment planner 16, in a non-limiting example, can develop the treatment plan in view of the preliminary ablation area. The treatment planner 16 can confirm the preliminary ablation area, refine the preliminary ablation area, and/or change the preliminary ablation area. The treatment planner 16 can develop the treatment plan with the goal of abolishing the arrhythmia by transecting critical limbs of the mechanism of action.

The system 10 can contain knowledge that can be exploited by the treatment planner 16 to develop the treatment plan. For example, the knowledge can be in the form of historical data that can be stored, for non-limiting example, in a database, in the non-transitory memory 8, and/or in a cloud computing environment (e.g., accessible to users/subscribers). The historical data can include stored historical mechanisms of action and treatment plans corresponding to the historical mechanisms of action. The historical mechanisms of action and corresponding treatment plans can be provided by users/subscribers and can be continually updated by the users/subscribers to aggregate the collective experience of the users/subscribers into a commonly accessible database. Additionally or alternatively, the historical data can include data mined or collected from one or more external sources regarding historical arrhythmia treatment plans and/or historical mechanism of action configurations. Moreover, the system 10 can include a learning function 4 that can analyze the historical information to aid in the development of the treatment plan.

The treatment planner 16 can develop the treatment plan according to the historical data and/or the learning function 4. The treatment planner 16, in a non-limiting example, can compare the plurality of electrograms, the activation map, and/or the proposed ablation area to one or more stored historical electrograms (e.g., in historical data). Based on the comparison, the treatment planner 16 can employ one or more pattern recognition techniques to determine a closest proxy from the plurality of stored historical electrograms. Examples of potential pattern recognition techniques include, but are not limited to: a classification algorithm, a regression algorithm, a sequence labeling algorithm, and a parsing algorithm. The treatment planner 16 can develop a proposed treatment plan based on the treatment plan of the closest proxy and offer the proposed treatment plan for approval (e.g., by displaying on the display 18 and receiving an input indicating approval or disapproval). Upon approval, the proposed treatment plan can be set as the treatment plan. If the approval is not received, the proposed treatment plan can be edited by the treatment planner 16 and a second proposed treatment plan can be presented for approval. The treatment plan and the corresponding activation map/electrocardiograms (and, optionally, an indication of success or lack of success of the treatment plan) can be stored in the historical data for future reference (e.g., to be used by the learning function 4).

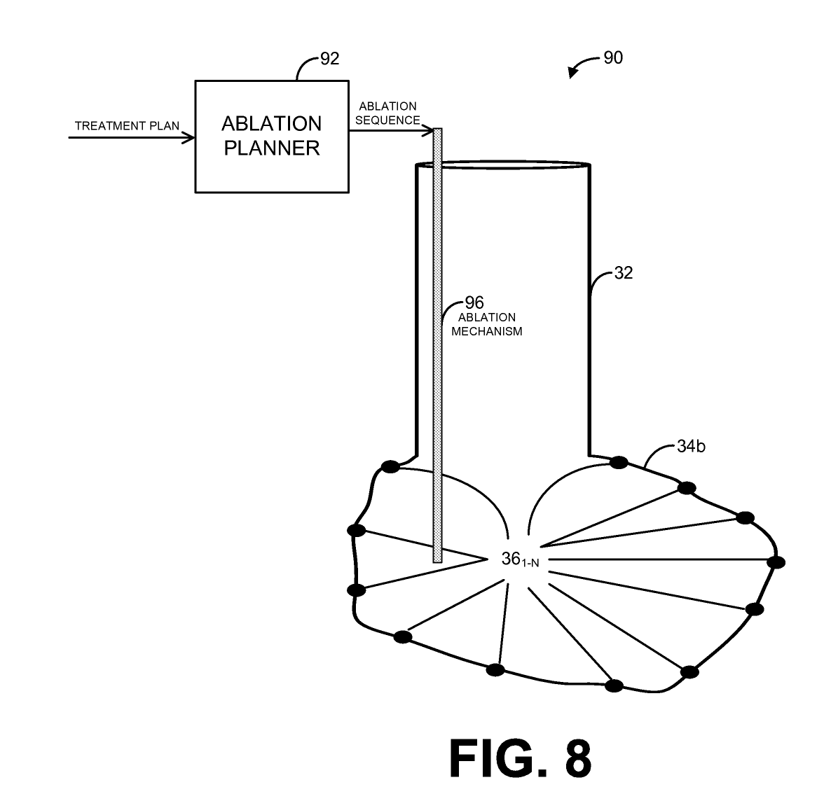

As a non-limiting example, as shown in FIG. 8, the modified catheter device 32 can include an ablation mechanism 96 (e.g., an ablation electrode capable of delivering ablative energy while minimizing adverse effects, such as tissue charring, thrombus formation, etc.) that is configured to ablate the driving circuit according to the ablation sequence. Although the ablation mechanism 96 is illustrated as a separate component within the modified catheter device 32, it will be understood that any of the electrodes 36_{1-N} can possess an ablation capability such that the ablation mechanism 96 is not separate from the expansion element 34a, 34b. Non-limiting examples of the type of energy that can be delivered by the ablation mechanism to ablate the mechanism of action can include: radio frequency (RF) energy, cryogenic energy, ultrasound energy, laser energy, or microwave energy.

As illustrated in FIG. 8, the treatment plan can be received by an ablation planner 92 (e.g., that is stored in memory and executable by a processor). The ablation planner 92 can determine an ablation sequence (e.g., one or more ablative lesion sets) to abolish the arrhythmia based on the treatment plan. For example, the treatment plan can include an ablation strategy that the ablation planner 92 can use to develop the ablation sequence. The ablation planner 92 can operate in connection with the treatment planner 16 to guide the ablation sequence so that the mechanism of action is ablated.

### IV. Methods

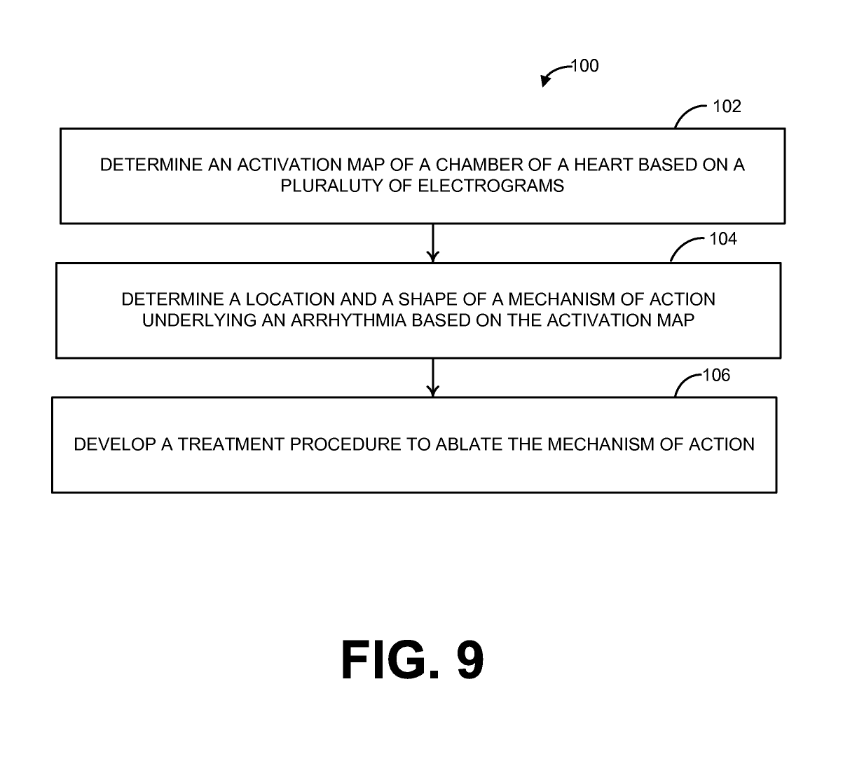

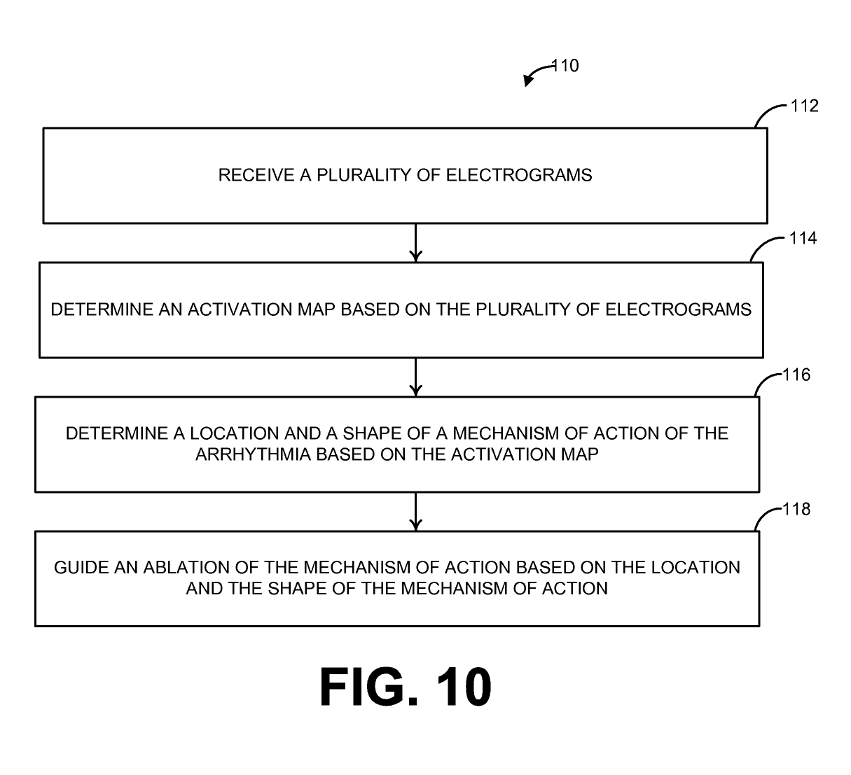

A second aspect of the present disclosure can include methods for developing a treatment plan to abolish an unstable arrhythmia. One example method 100 for developing a treatment plan to abolish a mechanism of action of an arrhythmia is shown in FIG. 9. Another example method 110 for identifying and treating a mechanism of action of an arrhythmia is shown in FIG. 10.

The methods 100 and 110 of FIGS. 9 and 10 are illustrated as process flow diagrams with flowchart illustrations. For purposes of simplicity, the method 100 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 100 and 110.

One or more blocks of the respective flowchart illustration of FIGS. 9 and 10, and combinations of blocks in the block flowchart illustrations, can be implemented by computer program instructions. These computer program instructions can be stored in memory and provided to a processor of a general purpose computer, special purpose computer, and/or other programmable data processing apparatus to produce a machine, such that the instructions, which execute via the processor of the computer and/or other programmable data processing apparatus, create mechanisms for implementing the steps/acts specified in the flowchart blocks and/or the associated description. In other words, the steps/acts can be implemented by a system comprising a processor that can access the computer-executable instructions that are stored in a non-transitory memory.

Referring to FIG. 9, an aspect of the present disclosure can include a method 100 for developing a treatment plan to abolish a mechanism of action of an arrhythmia in accordance with an aspect of the present disclosure. At 102, an activation map of a chamber of a heart can be determined (e.g., by conduction mapper 12) based on a plurality of electrograms. The activation map can reveal the mechanism of action of the arrhythmia. For example, the activation map can provide information related to electrical propagation velocity, information related to attenuation, information defining a zone of dead tissue, and/or information related to relative activation. In some instances, the activation map can be rendered as a visualization and displayed on a display device to illustrate the mechanism of action.

As an example, the electrograms can be recorded by a plurality of electrodes in contact with the wall of the chamber of the heart in response to an electrical perturbation to an area of the heart. The electrodes can be located on an expandable element of a catheter. The expandable element can be adapted to contact the chamber wall (e.g., made of a flexible material). In some instances, the activation map can be a dynamic activation map constructed in response to one or more electrical perturbations in one or more regions of the heart. The dynamic activation map can illustrate how an electrical impulse propagates through the chamber of the heart with time. For example, the electrical perturbation can include two or more pulses at different locations within the chamber or external to the chamber either at the same time or at different times. The method 100 can provide the ability of introducing a perturbation and collecting responses to the perturbation from points across the chamber simultaneously.

At 104, a location and a shape of the mechanism of action underlying the arrhythmia (e.g., ablation area) can be determined (e.g., by mechanism of action identifier 14) based on the activation map. At 106, a treatment plan to ablate the mechanism of action can be developed (e.g., by treatment planner 16). For example, the development of the treatment plan can be based on consulting a library of previous successful ablations of similar mechanisms of action (e.g., learning function 4).

Referring to FIG. 10, an aspect of the present disclosure can include a method 110 for identifying and treating a mechanism of action of an arrhythmia. At 112, a plurality of electrograms can be received (e.g., by conduction mapper 12). As an example, the electrograms can be recorded by a plurality of electrodes in contact with the wall of the chamber of the heart in response to an electrical perturbation to an area of the heart. The electrodes can be located on an expandable element of a catheter. The expandable element can be adapted to contact the chamber wall (e.g., made of a flexible material). The electrograms can be recorded in response to one or more electrical perturbations. For example, the electrical perturbation can include two or more pulses at different locations within the chamber or external to the chamber either at the same time or at different times (e.g., from pairs of electrodes within the plurality of electrodes). Similar to the method 100, the method 110 can provide the ability of introducing a perturbation and collecting responses to the perturbation from points across the chamber simultaneously.

At 114, an activation map can be determined (e.g., by conduction mapper 12) based on the plurality of electrograms. The activation map can reveal the mechanism of action of the arrhythmia. For example, the activation map can provide information related to electrical propagation velocity, information related to attenuation, information defining a zone of dead tissue, and/or information related to relative activation. In some instances, the activation map can be a dynamic activation map constructed in response to one or more electrical perturbations in one or more regions of the heart. The dynamic activation map can illustrate how an electrical impulse propagates through the chamber of the heart with time. For example, the electrical perturbation can include two or more pulses at different locations within the chamber or external to the chamber either at the same time or at different times.

At 116, a location and a shape of a mechanism of action of the arrhythmia can be determined (e.g., by mechanism of action identifier). At 118, an ablation of the mechanism of action can be guided (e.g., by treatment planner 16) based on the location and the shape of the mechanism of action. For example, the ablation can be guided using an imaging technique and/or based on the electrograms.

### V. Example Computer System

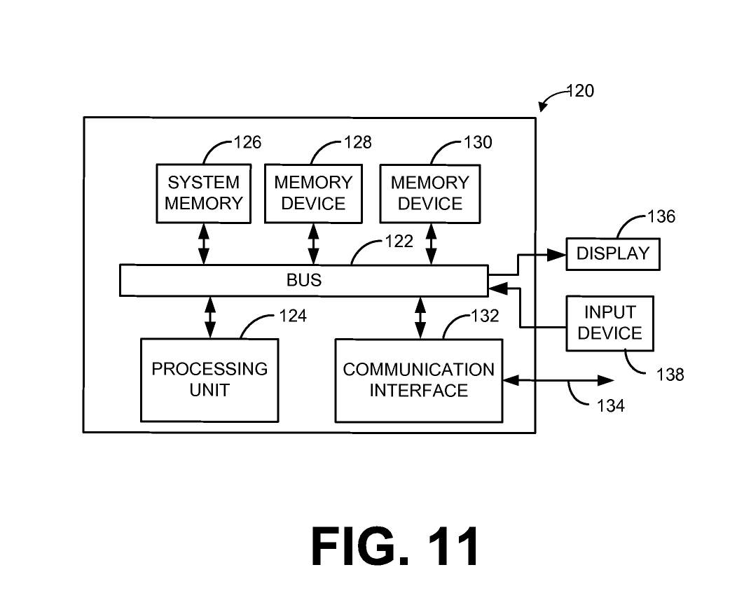

FIG. 11 is a schematic block diagram illustrating an exemplary system 120 of hardware components capable of implementing examples of the systems and methods of FIGS. 1 and 9-10. The system 120 can include various systems and subsystems, including a personal computer, a laptop computer, a workstation, a computer system, an appliance, an application-specific integrated circuit (ASIC), a server, a server blade center, a server farm, etc.

The system 120 can includes a system bus 122, a processing unit 124, a system memory 126, memory devices 128 and 130, a communication interface 132 (e.g., a network interface), a communication link 134, a display 136 (e.g., a video screen), and an input device 138 (e.g., a keyboard and/or a mouse). The system bus 122 can be in communication with the processing unit 124 and the system memory 126. The additional memory devices 128 and 130, such as a hard disk drive, server, stand alone database, or other non-volatile memory, can also be in communication with the system bus 122. The system bus 122 interconnects the processing unit 124, the memory devices 126-130, the communication interface 132, the display 136, and the input device 138. In some examples, the system bus 122 also interconnects an additional port (not shown), such as a universal serial bus (USB) port. The processing unit 124 can be a computing device that executes a set of instructions to implement the operations of examples disclosed herein. The processing unit 124 can include a processing core.

The memory devices 126, 128 and 130 can store data, programs, instructions, database queries in text or compiled form, and any other information that can be needed to operate a computer. The memory devices 126, 128 and 130 can be implemented as tangible computer-readable media (integrated or removable) such as a memory card, disk drive, compact disk (CD), or server accessible over a network. In certain examples, the memory devices 126, 128 and 130 can store data, including text data, image data, video data, and/or audio data, portions of which can be available in formats comprehensible to human beings. Additionally or alternatively, the system 120 can access an external data source or query source through the communication interface 132, which can communicate with the system bus 122 and the communication link 134.

In operation, the system 120 can be used to implement one or more parts of a system 10 of FIG. 1 that can identifies a mechanism of action of an arrhythmia and develops a treatment plan to abolish the mechanism of action. Computer executable logic for implementing the system 10 can reside on one or more of the system memory 126, and the memory devices 128, 130 in accordance with certain examples. The processing unit 124 can execute one or more computer executable instructions originating from the system memory 126 and/or the memory devices 128 and 130. The term “computer readable medium” as used herein refers to a medium that participates in providing instructions to the processing unit 124 for execution, and can, in practice, refer to multiple, operatively connected apparatuses for storing machine executable instructions.

From the above description, those skilled in the art will perceive improvements, changes and modifications. Such improvements, changes and modifications are within the skill of one in the art and are intended to be covered by the appended claims.

What is claimed is:

1. A catheter for insertion into a heart, comprising:
an elongated body;
a distal end of the body having an expansion element comprising a plurality of electrodes,
wherein the expansion element is configured to expand within a chamber of the heart in a shape corresponding to the shape of the chamber so that at least a portion of the plurality of electrodes contact a wall of the chamber to enable a characterization of a mechanism of action underlying an arrhythmia from an activation map created based on a plurality of electrograms recorded by the plurality of electrodes in response to an electrical perturbation to the chamber.

2. The catheter of claim 1, wherein the activation map is a dynamic activation map of the chamber that updates with time to illustrate a dynamic interplay between at least two driving circuits of the mechanism of action.

3. The catheter of claim 1, wherein the plurality of electrodes each contact the wall of the chamber.

4. The catheter of claim 1, wherein the plurality of electrograms are recorded during a small number of beats of the heart during the pacing sequence without requiring rotation of the expansion element.

5. The catheter of claim 1, wherein the mechanism of action is characterized by at least one of a size parameter and a location parameter.

6. The catheter of claim 1, wherein the plurality of electrodes comprises at least one pacing electrode pair configured to provide the perturbation to one or more areas of the heart.

7. The catheter of claim 1, wherein the perturbation is provided from a second chamber of the heart that is contralateral to the chamber.

8. The catheter of claim 1, wherein the expansion element comprises a nitinol frame that is connected by an actuating wire through the body to a control mechanism that activates an expansion of the expansion element or a contraction of the expansion element.

9. The catheter of claim 1, wherein the expansion element comprises an inflatable element and is expandable by inflation of the inflatable element with a fluid medium.

10. The catheter of claim 1, wherein the expansion element is configured to expand to a plurality of intermediate sizes to provide flexible sizing to match a plurality of sizes of a plurality of chambers of the heart in a plurality of patients.

11. The catheter of claim 1, wherein the expansion element is configured to be elastic and conformable to the chamber of the heart to minimize heart wall irritation.

12. The catheter of claim 1, further comprising an ablation mechanism configured to ablate the mechanism of action according to a treatment plan developed based on the characterization of the mechanism of action,
wherein the ablation mechanism is configured to deliver at least one of radio frequency (RF) energy, cryogenic energy, ultrasound energy, laser energy, or microwave energy to ablate the mechanism of action.

13. A method, comprising:
receiving, by a system comprising a non-transitory memory and a processor, a plurality of electrograms recorded by a plurality of electrodes contacting a wall of a chamber of the heart at a corresponding plurality of different locations within the chamber in response to an electrical perturbation to the chamber;
determining, by the system, an activation map of the chamber based on the plurality of electrograms;
determining, by the system, a location and a shape of a mechanism of action of the arrhythmia based on the activation map; and
guiding, by the system, an ablation of the mechanism of action based on the location and the shape of the mechanism of action.

14. The method of claim 13, further comprising rendering, by the system, a visualization of the activation map on a display device illustrating the mechanism of action.

15. The method of claim 13, wherein the activation map changes dynamically in response to the electrical perturbation.

16. The method of claim 15, wherein the electrical perturbation comprises at least two electrical perturbations at different locations within the chamber.

17. The method of claim 13, wherein the activation map provides at least one of information related to electrical propagation velocity, information related to attenuation, information defining a zone of dead tissue, and information related to relative activation.

18. A system, comprising:
a non-transitory memory to store machine-executable instructions; and
a processor to access the non-transitory memory and execute the machine-executable instructions to cause a computing device to perform operations, the operations comprising:
receive a plurality of electrograms recorded by a plurality of electrodes contacting a wall of a chamber of the heart at a corresponding plurality of different locations within the chamber in response to an electrical perturbation to the chamber;
determine an activation map of the chamber based on the plurality of electrograms;
determine a location and a shape of a mechanism of action of the arrhythmia based on the activation map; and
guide an ablation of the mechanism of action based on the location and the shape of the mechanism of action.

19. The system of claim 18, wherein the activation map changes dynamically in response to the electrical perturbation,
wherein the electrical perturbation comprises at least two electrical perturbations at different locations within the chamber.

20. The system of claim 18, wherein the activation map provides at least one of information related to electrical propagation velocity, information related to attenuation, information defining a zone of dead tissue, and information related to relative activation.
